(19) United States
(12) Patent Application Publication
(10) Pub. No.: US 2020/0113452 A1
(43) Pub. Date: Apr. 16, 2020

(54) ACTIVE BIOMONITOR

(71) Applicant: Jose Carmelo Martinez, Toms River, NJ (US)

(72) Inventor: Jose Carmelo Martinez, Toms River, NJ (US)

(21) Appl. No.: 16/707,034

(22) Filed: Dec. 9, 2019

Related U.S. Application Data
(63) Continuation-in-part of application No. 15/353,125, filed on Nov. 16, 2016, now abandoned.

(51) Int. Cl. A61B 5/0205; A61B 5/01; A61B 5/1455; A61B 5/00; A61B 5/0295; A61B 5/11

(52) U.S. Cl. CPC A61B 5/02055; A61B 5/01; A61B 5/14552; A61B 5/0022; A61B 5/6833; A61B 5/02438; A61B 5/7405; A61B 5/747; A61B 5/7475; A61B 5/0295; A61B 5/1112; A61B 5/742

(57) ABSTRACT

A biomonitor having various functionality for actively monitoring certain vital signs and wellness data is provided. The biomonitor is adapted to compare the wellness data to programmed and/or determined thresholds and call 911 if such wellness data indicates a medical emergency.

### FIELD OF THE INVENTION

The present invention relates to health monitoring devices and, more particularly, to a biomonitor providing various functionality for actively monitoring certain vital signs and wellness data.

### CROSS-REFERENCE TO RELATED APPLICATION

The present application is a continuation-in-part (CIP) of and claims priority from non-provisional patent application Ser. No. 15/353,125, entitled ACTIVE BIOMONITOR, filed on Nov. 16, 2016 the contents of which are hereby incorporated by reference into this application.

### BACKGROUND OF THE INVENTION

Sudden Infant Death Syndrome (SIDS) is the leading cause of death in infant newborns 12-months of age. In 2010 over two thousand infants passed away from SIDS. On the average a child who has died from SIDS will show signs of aspirating or choking. Accidental suffocations, claims another 665 infants per year. Aside from this you have infants and children with asthma, epilepsy, diabetes and other congenital diseases that need constant monitoring but cannot be admitted to a hospital. Add to this the fact that many parents cannot afford a stay at home nurse 24/7. Many infants and child congenital diseases manifest as a compromised airway, causing a dramatic change in the child's heart rate and oxygen level (cardiac arrest is usually a result of respiratory arrest in a child).

Current monitors only passively monitor vital signs, but are unable to actively assist in responding to a medical emergency the vital signs indicate.

As can be seen, there is a need for an improved biomonitor for children so as to actively monitoring certain vital signs.

### BRIEF DESCRIPTION OF THE DRAWINGS

FIG. 1 is a schematic view of an exemplary embodiment of the present invention;

FIG. 2 is a perspective view of an exemplary embodiment of the present invention; and

FIG. 3 is a flowchart of an exemplary embodiment of the present invention.

FIG. 4 is a schematic diagram of components of an exemplary embodiment of the present invention.

### SUMMARY OF THE INVENTION

In one aspect of the present invention, a biomonitor providing various functionality for actively monitoring certain vital signs and wellness data includes a body housing a plurality of wellness sensors configured for measuring a plurality of vital signs of a human user; a band connected to the body, wherein the band is adapted for securing the body to the human user so that the plurality of wellness sensors operably engages the human user; a microprocessor coupled to the plurality of wellness sensors, wherein the microprocessor is configured to categorize the plurality of vital signs in one of four categories including critical, unstable, potential, and stable; and a software application loaded on the microprocessor, wherein the software application is configured to automatically call 911 if the plurality of vital signs is categorized as critical or unstable.

In another aspect of the present invention, the biomonitor providing various functionality for actively monitoring certain vital signs and wellness data includes a body housing a plurality of wellness sensors configured for measuring a plurality of vital signs of a human user; the plurality of wellness sensors includes a tri-sensor pulse oximetry sensor, at least one temperature sensor, and a microphone sensor; a band connected to the body, wherein the band is adapted for securing the body to the human user so that the plurality of wellness sensors operably engages the human user; a microprocessor coupled to the plurality of wellness sensors, wherein the microprocessor is configured to categorize the plurality of vital signs in one of four categories including critical, unstable, potential, and stable; a speaker coupled to the microprocessor; a display screen coupled to the microprocessor; a LED light coupled to the microprocessor; a GPS coupled to the microprocessor, wherein the GPS is configured to define a physical location of the device; a software application loaded on the microprocessor, wherein the software application is configured to automatically call 911 if the plurality of vital signs is categorized as critical or unstable through a three-tone prompt transmitting the physical location of the device, the plurality of vital signs of the human user, and the categorical identification of the plurality of vital signs; and the microprocessor configured to: output through the speaker and or the display screen at least one recorded instruction tailored to at least one medical need of the human user; and activate the LED light when automatically calling 911.

These and other features, aspects and advantages of the present invention will become better understood with reference to the following drawings, description and claims.

### DETAILED SPECIFICATION

The following detailed description is of the best currently contemplated modes of carrying out exemplary embodiments of the invention. The description is not to be taken in a limiting sense, but is made merely for the purpose of illustrating the general principles of the invention, since the scope of the invention is best defined by the appended claims.

Broadly, an embodiment of the present invention provides a biomonitor providing various functionality for actively monitoring certain vital signs and wellness data, wherein the biomonitor is adapted to compare the wellness data to programmed and/or determined thresholds and call 911 if such wellness data indicates a medical emergency.

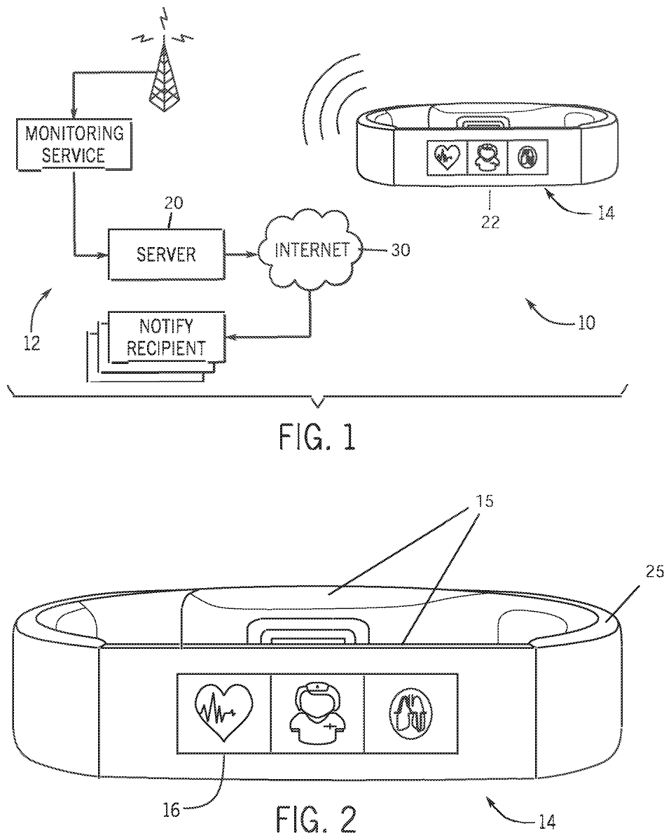

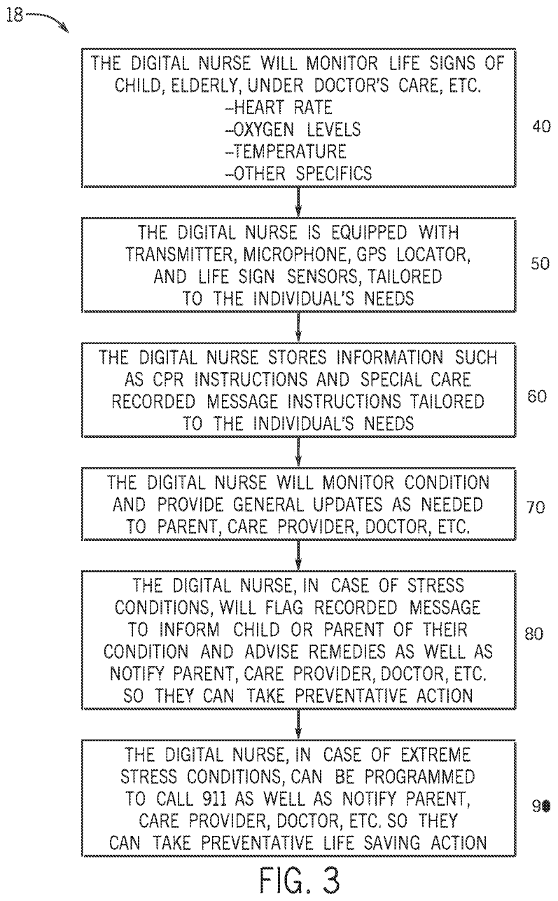

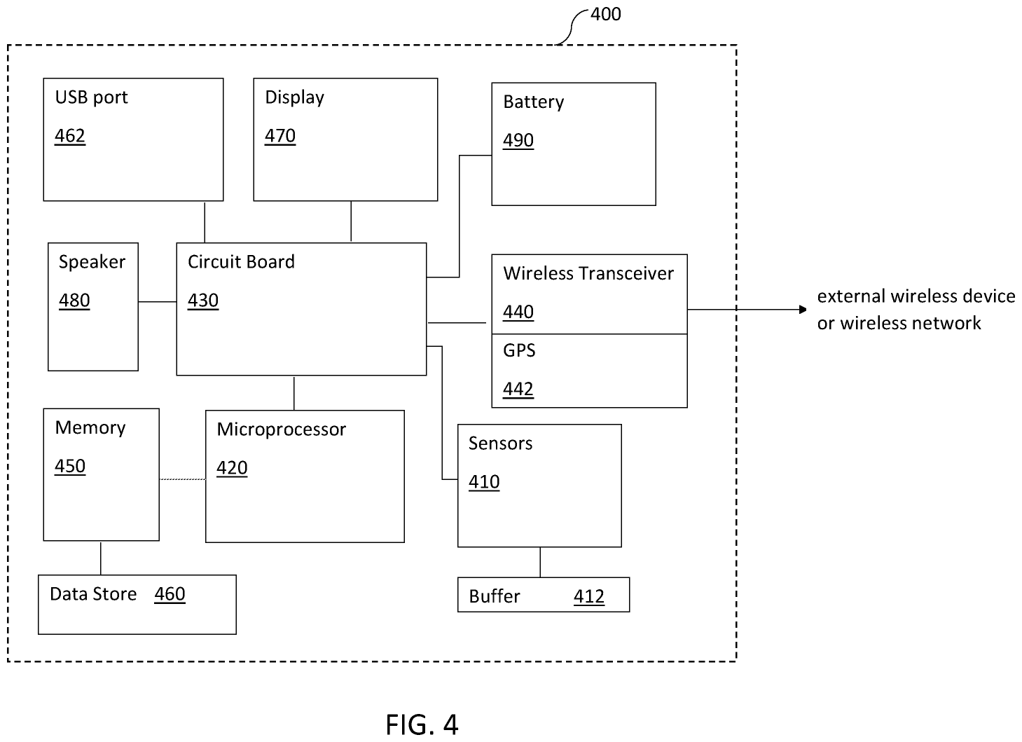

Referring to FIG. 1, the present invention may include at least one computer with a user interface. The computer may include at least one processing unit coupled to a form of memory including, but not limited to, a computing device, a server 20, a microprocessor, a desktop, a laptop, and smart device, such as a tablet, a smart phone, smart watch, a smart bracelet 22, or the like. The computer may include a program product including a machine-readable program code for causing, when executed, the computer to perform steps. The program product may include software which may either be loaded onto the computer or accessed by the computer. The loaded software may include an application on a smart device. The software may be accessed by the computer using a web browser. The computer may access the software via the web browser using the internet 30, extranet, intranet, host server, internet cloud, wifi network, and the like.

Referring to FIG. 2, the present invention may include a biomonitor 10 for detecting life threats and actively alerting assistance to said life threats. The present invention may be called the “Digital Nurse”. In certain embodiments, the biomonitor 10 may be designed to detect life threats to children with congenital diseases and to automatically call for the appropriate resources to help save the child. The biomonitor 10 may provide a body (or bodies) 15 connected to a wrist band 25 dimensioned and adapted to secure the biomonitor 10 around the wrist of a user. By constantly monitoring vital signs, such as their temperature, heart rate, blood pressure, respiratory rate and pulse and oxygen level, and whether there is a marked or critical change in said vital signs, the biomonitor 10 is able to react to a medical emergency of the user. If the vitals reach a dangerous or critical level, then the biomonitor 10 is adapted to take the appropriate action to alert the necessary resources, as well as be adapted to provide give onsite CPR instruction to those on the scene. Upon detecting a child in cardiac or respiratory arrest, for example, the biomonitor 10 with its “Auto 911” feature calls for an ambulance, such as by calling 911, and through the call provides the child's name, present location, condition and last set of vital signs taken.

The present invention may include the following primary components 14: a microprocessor, such as the MCP2515, with sufficient memory and firmware and a software application, Bluetooth functionality, GPS functionality, an output signal, such as an LED light, a power source, a user interface 16, a plurality of wellness sensors including a tri-sensor pulse oximetry sensor; at least one temperature sensor; and a microphone sensor, as well as other wellness sensors adapted for the following: in cancer patients the detection of new cancer cells or the remission of existing ones for better monitoring and follow up treatment; detection of central nervous system or synapse irregularities to treat or prevent seizures; detection of rising vascular pressure to treat or prevent Cerebral Vascular Accidents or Transient lschemic Attacks (Stroke patients); detection of rising Systolic/Diastolic pressure to treat or prevent Myocardial Infarctions (Heart Attacks); and detection of rising or dropping glycogen or insulin levels for the treatment of diabetics.

The above-mentioned wellness sensors, functionality, power source and output signal may be coupled to the microprocessor so as to be controlled by the software application for measuring wellness data. These primary components 14 may be encased in the body 15 of the biomonitor 10.

The biomonitor 10 may be made of a hyper allergenic waterproof material and may come in a variety of colors and designs. The bracelet may be flexible and comfortable to accommodate a child's movement. Along the body 15, the wellness sensors may be disposed for interfacing with skin of the wearer-user.

The software application may be adapted to provide the following features: Multi-language capability (using Google® translate); Auto GPS (using Google® Maps); Auto Temperature read; Auto Vital Signs read; Auto CPR instruction based on AMA and AHA standards; Auto C.U.P.S. status—based on the numerical values of the vital signs the app may place the child in one of four categories (Critical, Unstable, Potential Unstable, Stable); and Auto 911 with message.

The biomonitor 10 enables a parent to monitor the heart rate, oxygen level and temperature in a child and alerts the parent in case there is an emergent change in these readings. Each may include the wellness sensors that may continuously monitor the heart rate, oxygen level and temperature in both infants and children. The readings are displayed in number values that is easy for any parent to read and understand. The unit may also include a built-in microphone so that the parent can hear the child. The biomonitor 10 may also work with the software application that may be compatible with smart devices, including but not limited to smart phones, laptops, desktops, tablets and smart TV's. Each unit can be personalized to a child's name, date of birth, and weight. For example, through user interface 16, a user can navigate the onscreen instructions to fill in the user's personal information, medical condition, related medications and allergies, via prompts, multiple choice answers, or the like. Based on this information the unit may then calibrate to the child's proper heart rate and oxygen level.

The microprocessor may provide an alarm system 12 adapted to analyze the wellness data so that, in one embodiment, the microprocessor generates alarms if the value of any of the wellness data exceeds predetermined thresholds, wherein said thresholds are likely established by medical professionals. In such a case, microprocessor may generate exportable messages, for example phone messages, email messages and SMS messages sent to user-specified addresses, such as 911. Biomonitor 10 may be adapted to activate the output signal. The alarm system may be coupled with the GPS, WiFi, and GTS so as the above-mentioned alarm message (911 call, SMS or email) includes the user's current location even if the child is unconscious.

If the biomonitor 10 detects a Critical or Unstable level in the child's vital signs 911 is automatically called. A three-tone prompt may let the 911 receiving operator know that the call is from the biomonitor 10. The message may include the child's name, present location and the status of the child (Cardiac Arrest or Respiratory Arrest) and last recorded set of vital signs. The message may repeat itself until the 911 operator disengages the call. Auto 911 messages may be delivered on in English or other languages. The output signal may blink in RED and White continuously when Auto 911 is activated. This may also aid in the recovery of a child in the event that the child is lost in a low-lit area or in a mass crowd. Through the Auto CPR feature, the biomonitor 10 may also be able to give directions on either Rescue Breathing or CPR as per the American Heart Association and American Medical Association Guidelines. This can be done by hitting the Auto CPR icon on the phone or by pressing the biomonitor 10 face on the monitor.

The above features can be activated by the parent by checking off the appropriate boxes. The components may detect critical changes in heart, stroke, diabetic, cancer, and epileptic patients. The configuration can also be applied to patients who are already admitted to a hospital, hospice or nursing home for total and complete monitoring of the patient.

A method of using the present invention may include the following. The biomonitor 10 disclosed above may be provided. A user may secure the biomonitor 10 about the wrist of a child after programming the microprocessor to set the thresholds associated with each wellness sensor.

Additionally, for the elderly the biomonitor 10 may erase the fear of living alone by providing the confidence that if the user's medical condition should for whatever reason put them in a critical situation that the biomonitor 10 is not just there to monitor but to make that much needed 911 call to get them the help they desperately need. Better than a medical alert bracelet or any system that requires activation and verbal response the Digital Nurse will have all the necessary information stored and ready for all necessary resources that need it. This is especially critical if the elderly patient should fall unconscious due to illness or injury. In the nursing home setting where there is limited staffing and so many residents the biomonitor 10 is indispensable. Its constant monitoring of the patient will give family members peace of mind as they are able to see their loved one's present physical state.

In a hospital setting the biomonitor 10 applications can be numerous. First it acts as a digital ID band for each patient. Second its monitoring system can be implemented into the facilities computer network giving doctors immediate on sight data of each patient. Third nurses can leave the mundane task of taking vital signs and temperatures. The biomonitor 10 with its constant monitoring of these vital systems will free nurses to do more important patient related tasks and at the same time be more readily available to assist doctors in more serious medical issues. This is especially ideal for a facility with limited medical staffing or if tending to a major catastrophe or event. Lastly for those patients who are highly contagious and who have to be separated or quarantined the biomonitor 10 will reduce the risk of exposure to hospital workers for reasons of vital signs and temperature taking.

Also, the biomonitor 10 can be implemented by both the Center for Disease Control and The World Health Organization in areas where there might be an outbreak of Ebola, Swine Flu, Malaria, Leprosy and Avian Influenza. By limiting contact between the health care workers and those infected the spread of infection is thereby limited. All this while still maintaining a constant watch of every patients vital functions and temperature. On a large scale like this not a single patient will be lost in the shuffle. The biomonitor 10 can do the job of monitoring these patients while giving the medical staff the freedom to treat the more serious, once again minimizing cost of staffing and still saving lives.

In method 18, beginning with step 40, the digital nurse (biomonitor 10) may monitor life signs of the wearer (child, elderly, anyone under a medical professional's care), including their heart rate, oxygen levels, temperature, and other specifics. In step 50, the biomonitor 10 may be equipped with a transmitter, microphone, GPS locator, and life sign/wellness sensors tailored to the user's medical needs. In step 60, the biomonitor 10 may store information such as CPR instructions and special care recorded message instructions tailored to the user's medical needs. In step 70, the biomonitor 10 may monitor wellness data/conditions of the user, providing general updates as needed to parents, care providers, medical professionals and the like. In step 80, the biomonitor 10 in the case of stress conditions may flag recorded message to inform child or parent of their condition and advise remedies as well as notify parents, care provider, medical professionals and the like so they can take preventative action. In step 90, the biomonitor 10 in case of extreme stress conditions can be programmed to call 911 as well as notify parents, care provider, medical professionals and the like so they can take preventative life saving action.

According to another embodiment, a biomonitor 400 is provided having a housing in the form of a wearable patch configured to attach to a pulse point of the human body. The housing includes an adhesive hydrocolloid base and a plurality of sensors 410 housed therein or integrated into a surface of the housing. The sensors 410 are configured to acquire vital sign data including at least the following vital signs: (1) heart rate, (2) oxygen level, and (3) body temperature of a user wearing the patch device. The terms “sensors” and “biosensors” shall be regarded as equivalent terms throughout this application.

The biomonitor 400 includes a microprocessor 420 connected to a circuit board 430 mounted inside the housing which is coupled to the plurality of sensors. The microprocessor 420 is configured to receive and evaluate the vital sign data in order to classify each of the vital signs as critical, unstable, potential, or stable, as already discussed above. The microprocessor 420 may be a member of the ARM11 family of core processors, such as the ARM1176JZ (F)-S processor. The microprocessor may also be an ARM Cortex-A or ARM Cortex-R core processor. One of ordinary skill in the art will recognize that any suitable processor may be utilized for the biomonitor. Furthermore, although the biomonitor is described as having a patch housing, the housing could have a different form such as a band (e.g., arm band, wrist band, leg band, head band, etc.) or any other form, and may be embedded in an article of clothing.

In a preferred embodiment, the circuit board and circuits connected thereto are flexible printed circuits and circuit board, such as the flex printed circuits/circuit board sold by Flexible Circuit Technologies of Minneapolis, Minn. Similarly, the sensors may be flexible printed biosensors such as the flexible biosensors manufactured by Butler Technologies Inc. of Butler, Pa. or those manufactured by Zimmer & Peacock of Napa, Calif. The biomonitor patch is thus able to flexibly conform to the part of the body to which it is attached.

According to one embodiment, at least one of the sensors may be a pressure sensor based on conductive single-walled carbon nanotube (SWCNT) and/or alginate hydrogel spheres. Another at least one sensor may be a pulse oximeter with plethysmography configured to acquire blood oxygen levels and detect blood volume changes. Another at least one sensor may be a HSPPA pressure sensor, such as the ALPS Electric HSPPA Board Mount Pressure Sensor manufactured by ALPS ELECTRIC, INC., with its U.S. headquarters in Santa Clara, Calif. Another of the sensors may be a temperature sensor configured to acquire ambient temperature and skin temperature of a user wearing the biomonitor. A memory buffer 412 may be provided to temporarily store sensor data.

The biomonitor of the present invention may further include a GPS device 442 mounted inside the housing. The GPS may be used to track the location of the user. Alternatively, the biomonitor device may be tracked using a cellular network or an IP address of the biomonitor device. The biomonitor patch may also include a homing device that allows emergency personnel to locate the device. One of ordinary skill in the art will recognize that different technologies may be employed to track and locate the biomonitor device.

In another embodiment, the biomonitor includes a micro SIM (subscriber identity module) card connected to the circuit board to store information used to access a network. In addition, the biomonitor device may include LED lights that pulsate in one or more colors, such as red, yellow, green, blue, white, etc. when a vital sign is critical or unstable, if the vital signs are outside of a predetermined normal range, or after activating the 911 call function. The terms “biomonitor” and “biomonitor device” shall be regarded as interchangeable terms.

The biomonitor further includes a communication means 440 configured to send and receive data over a network. The type of communication can include, for example, Internet, telecommunications, radio communications, and satellite communications and the like. The communication means includes data communications equipment (DCE), such as a network interface card, Wi-Fi PCI or PCI Express card, Dual Band Wireless network adapter (e.g., Intel Dual Band Wireless-AC 7260 Wi-Fi and Bluetooth combination chip), blue tooth module, Wi-Fi module, transceiver, or other communication device may be used for sending and receiving data over a network. As discussed above, the wireless card or chip is preferably a printed flex card or chip. However, one of ordinary skill in the art will recognize that the chips, memory, sensors, communication means, display, circuit board, and biomonitor housing can be made of either rigid, flexible, or a combination of rigid and flexible material.

The biomonitor further includes one or more memory modules 450 coupled to the microprocessor. The memory module can include ROM, RAM (e.g. SRAM and/or DRAM), a memory buffer, or the like. In addition, a storage device 460 may be built into or mounted on the circuit board in the biomonitor device. A storage device, such as a micro storage device may also be connected to the biomonitor device by plugging the storage device into a memory slot in the biomonitor device, such as a universal serial bus (USB) slot 462. The amount of RAM memory installed in the biomonitor may vary, but preferably has a capacity of at least 4 GB+512 MB.

A display 470 is integrated into a face of the biomonitor housing and is configured to display at least some of the vital signs sensed by the plurality of sensors. The display is preferably a flexible display, such as a flexible OLED display. The vital sign data displayed on the display may be displayed in any suitable color and may flash intermittently or be displayed in a continuous mode. In addition, the displayed vital sign data may be accompanied by an audio recording stating the vital signs. The audio recording may come on automatically when a vital sign is out of a predetermined normal range, or could be provided in response to user input, such as pressing a push button or selecting a touch screen button object.

A speaker 480 integrated into the housing provides audio output such as the audio vital signs or other audio alerts. In addition, the biomonitor device may be powered by a small battery 490, such as a CR 2025/CTL 1025-3V battery, or one or more small photovoltaic cells or micro solar panels. A combination of one or more batteries and micro solar panels may also be utilized.

In one embodiment, the 911 call function generates a 3-tone prompt when a 911 operator answers the call. The 3-tone prompt is then followed by an automated message that provides the name, present location, and status of the user wearing the biomonitor patch. This allows emergency personnel to quickly be dispatched to the user's location to provide emergency assistance.

The biomonitor of the present invention is further configured to display a barcode image on its digital display. Specifically, the biomonitor has barcode data stored in memory and the microprocessor is configured to display the barcode image in response to a user input. The user input might include selecting a barcode button object on a touch screen, pressing a push button on the biomonitor device, or issuing a voice command. In the case of voice commands, the biomonitor is configured to recognize certain voice commands as input for the purpose of generating a predetermined output such as displaying the barcode image. One of ordinary skill in the art will appreciate that a user input can include tactile interaction with a touchscreen, pressing a push button, issuing a voice command, or using a peripheral input device such as a stylus, mouse, keyboard, or the like.

According to an embodiment of the invention, computer readable instructions in the form of a software program reside in memory. When the computer readable instructions are executed by the microprocessor the microprocessor evaluates the vital sign data and, when at least one of the vital signs is classified as critical or unstable the microprocessor: (1) activates a 911 call function, where the 911 call function is configured to call 911 to alert emergency personnel of a health emergency at the location of the biomonitor; and (2) outputs audio instructions for administering potentially lifesaving intervention through the speaker. The audio instructions may be instructions for administering CPR and may be generated in response to receiving input from someone who is ready to administer CPR, such as receiving touch screen input when a person taps a button on the biomonitor patch that symbolizes CPR instructions.

Traditionally, a computer program consists of a finite sequence of computational instructions or program instructions. It will be appreciated that a programmable apparatus (i.e., computing device) can receive such a computer program and, by processing the computational instructions thereof, produce a further technical effect.

A programmable apparatus includes one or more microprocessors, microcontrollers, embedded microcontrollers, programmable digital signal processors, programmable devices, programmable gate arrays, programmable array logic, memory devices, application specific integrated circuits, or the like, which can be suitably employed or configured to process computer program instructions, execute computer logic, store computer data, and so on. Throughout this disclosure and elsewhere a computer can include any and all suitable combinations of at least one general purpose computer, special-purpose computer, programmable data processing apparatus, processor, processor architecture, and so on.

It will be understood that a computer can include a computer-readable storage medium and that this medium may be internal or external, removable and replaceable, or fixed. It will also be understood that a computer can include a Basic Input/Output System (BIOS), firmware, an operating system, a database, or the like that can include, interface with, or support the software and hardware described herein.

Embodiments of the system as described herein are not limited to applications involving conventional computer programs or programmable apparatuses that run them. It is contemplated, for example, that embodiments of the invention as claimed herein could include an optical computer, quantum computer, analog computer, or the like.

Regardless of the type of computer program or computer involved, a computer program can be loaded onto a computer to produce a particular machine that can perform any and all of the depicted functions. This particular machine provides a means for carrying out any and all of the depicted functions.

Any combination of one or more computer readable medium(s) may be utilized. The computer readable medium may be a computer readable signal medium or a computer readable storage medium. A computer readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer readable storage medium would include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a computer readable storage medium may be any tangible medium that can contain, or store a program for use by or in connection with an instruction execution system, apparatus, or device.

Computer program instructions can be stored in a computer-readable memory capable of directing a computer or other programmable data processing apparatus to function in a particular manner. The instructions stored in the computer-readable memory constitute an article of manufacture including computer-readable instructions for implementing any and all of the depicted functions.

A computer readable signal medium may include a propagated data signal with computer readable program code embodied therein, for example, in baseband or as part of a carrier wave. Such a propagated signal may take any of a variety of forms, including, but not limited to, electromagnetic, optical, or any suitable combination thereof. A computer readable signal medium may be any computer readable medium that is not a computer readable storage medium and that can communicate, propagate, or transport a program for use by or in connection with an instruction execution system, apparatus, or device.

Program code embodied on a computer readable medium may be transmitted using any appropriate medium, including but not limited to wireless, wireline, optical fiber cable, RF, etc., or any suitable combination of the foregoing.

The elements depicted in flowchart illustrations and block diagrams throughout the figures imply logical boundaries between the elements. However, according to software or hardware engineering practices, the depicted elements and the functions thereof may be implemented as parts of a monolithic software structure, as standalone software modules, or as modules that employ external routines, code, services, and so forth, or any combination of these. All such implementations are within the scope of the present disclosure.

In view of the foregoing, it will now be appreciated that elements of the block diagrams and flowchart illustrations support combinations of means for performing the specified functions, combinations of steps for performing the specified functions, program instruction means for performing the specified functions, and so on.

It will be appreciated that computer program instructions may include computer executable code. A variety of languages for expressing computer program instructions are possible, including without limitation C, C++, Java, JavaScript, assembly language, Lisp, and so on. Such languages may include assembly languages, hardware description languages, database programming languages, functional programming languages, imperative programming languages, and so on. In some embodiments, computer program instructions can be stored, compiled, or interpreted to run on a computer, a programmable data processing apparatus, a heterogeneous combination of processors or processor architectures, and so on.

In some embodiments, a computer enables execution of computer program instructions including multiple programs or threads. The multiple programs or threads may be processed more or less simultaneously to enhance utilization of the processor and to facilitate substantially simultaneous functions. By way of implementation, any and all methods, program codes, program instructions, and the like described herein may be implemented in one or more thread. The thread can spawn other threads, which can themselves have assigned priorities associated with them. In some embodiments, a computer can process these threads based on priority or any other order based on instructions provided in the program code.

Unless explicitly stated or otherwise clear from the context, the verbs “execute” and “process” are used interchangeably to indicate execute, process, interpret, compile, assemble, link, load, any and all combinations of the foregoing, or the like. Therefore, embodiments that execute or process computer program instructions, computer-executable code, or the like can suitably act upon the instructions or code in any and all of the ways just described.

The functions and operations presented herein are not inherently related to any particular computer or other apparatus. Various general-purpose systems may also be used with programs in accordance with the teachings herein, or it may prove convenient to construct more specialized apparatus to perform the required method steps. The required structure for a variety of these systems will be apparent to those of skill in the art, along with equivalent variations. In addition, embodiments of the invention are not described with reference to any particular programming language. It is appreciated that a variety of programming languages may be used to implement the present teachings as described herein, and any references to specific languages are provided for disclosure of enablement and best mode of embodiments of the invention. Embodiments of the invention are well suited to a wide variety of computer network systems over numerous topologies. Within this field, the configuration and management of large networks include storage devices and computers that are communicatively coupled to dissimilar computers and storage devices over a network, such as the Internet.

The functions, systems and methods herein described could be utilized and presented in a multitude of languages. Individual systems may be presented in one or more languages and the language may be changed with ease at any point in the process or methods described above. One of ordinary skill in the art would appreciate that there are numerous languages the system could be provided in, and embodiments of the present invention are contemplated for use with any language.

While multiple embodiments are disclosed, still other embodiments of the present invention will become apparent to those skilled in the art from this detailed description. The invention is capable of myriad modifications in various obvious aspects, all without departing from the spirit and scope of the present invention. Accordingly, the drawings and descriptions are to be regarded as illustrative in nature and not restrictive.

The computer-based data processing system and method described above is for purposes of example only, and may be implemented in any type of computer system or programming or processing environment, or in a computer program, alone or in conjunction with hardware. The present invention may also be implemented in software stored on a computer-readable medium and executed as a computer program on a general purpose or special purpose computer. For clarity, only those aspects of the system germane to the invention are described, and product details well known in the art are omitted. For the same reason, the computer hardware is not described in further detail. It should thus be understood that the invention is not limited to any specific computer language, program, or computer. It is further contemplated that the present invention may be run on a stand-alone computer system, or may be run from a server computer system that can be accessed by a plurality of client computer systems interconnected over an intranet network, or that is accessible to clients over the Internet. In addition, many embodiments of the present invention have application to a wide range of industries. To the extent the present application discloses a system, the method implemented by that system, as well as software stored on a computer-readable medium and executed as a computer program to perform the method on a general purpose or special purpose computer, are within the scope of the present invention. Further, to the extent the present application discloses a method, a system of apparatuses configured to implement the method are within the scope of the present invention.

It should be understood, of course, that the foregoing relates to exemplary embodiments of the invention and that modifications may be made without departing from the spirit and scope of the invention as set forth in the following claims.

1. A biomonitor patch, comprising:
a housing in the form of a patch configured to attach to a pulse point of the human body, said housing having an adhesive hydrocolloid base and comprising a plurality of sensors housed therein, wherein said plurality of sensors are configured to acquire vital sign data comprising the following vital signs: (1) heart rate, (2) oxygen level, and (3) body temperature of a user wearing the patch device;
a microprocessor connected to a circuit board mounted inside of said housing and coupled to the plurality of sensors, wherein said microprocessor is configured to receive and evaluate said vital sign data in order to classify each of the vital signs as critical, unstable, potential, or stable;
a communication means configured to send and receive data over a network;
a memory coupled to said microprocessor;
a digital display integrated into a face of said housing and configured to display at least some of said vital signs sensed by said plurality of sensors;
a speaker integrated into said housing;
computer readable instructions residing in said memory, wherein when said computer readable instructions are executed by said microprocessor said microprocessor evaluates said vital sign data and, when at least one of the vital signs are classified as critical or unstable said microprocessor (1) activates a 911 call function, wherein said 911 call function is configured to call 911 to alert emergency personnel of a health emergency at the location of the patch device, and (2) outputs audio instructions for administering potentially lifesaving intervention through said speaker.

2. The biomonitor patch of claim 1, wherein said memory has barcode data stored thereon that is used to display a barcode image on said digital display, wherein said microprocessor is configured to display said barcode image in response to a user input.

3. The biomonitor patch of claim 1, wherein the 911 call function generates a 3 tone prompt followed by an automated message that provides the name, present location, and status of the user wearing the diagnostic health patch when a 911 operator answers the 911 call.

4. The biomonitor patch of claim 1, wherein at least one of the plurality of sensors is a pressure sensor based on at least one of conductive single-walled carbon nanotube (SWCNT) and alginate hydrogel spheres.

5. The biomonitor patch of claim 1, wherein at least one of the plurality of sensors is a pulse oximeter with plethysmography configured to acquire blood oxygen levels and detect blood volume changes.

6. The biomonitor patch of claim 1, wherein at least one of the plurality of sensors is a HSPPA pressure sensor.

7. The biomonitor patch of claim 1, wherein at least one of the plurality of sensors is a temperature sensor configured to acquire ambient temperature and skin temperature.

8. The biomonitor patch of claim 1, further comprising a GPS device mounted in said housing.

9. The biomonitor patch of claim 1, further comprising a combination Wi-Fi and Bluetooth chip connected to said circuit board.

10. The biomonitor patch of claim 1, further comprising a micro SIM (subscriber identity module) card connected to said circuit board.

11. The biomonitor patch of claim 1, further comprising LED lights.

12. The biomonitor patch of claim 1, wherein the microprocessor is an ARM1176JZ (F)-S processor.

13. The biomonitor patch of claim 1, wherein said memory comprises one or more of a buffer, RAM and ROM.

14. The biomonitor patch of claim 13, wherein said RAM has a memory capacity of at least 4 GB+512 MB.

15. The biomonitor patch of claim 1, further comprising a battery.
